(19) United States
(12) Patent Application Publication
(10) Pub. No.: US 2006/0182807 A1
(43) Pub. Date: Aug. 17, 2006

(54) Copolymer and hemoprotein based novel compounds and uses thereof

(76) Inventors: Christine Vauthier, BURES-SUR-YVETTE (FR); Cedric Chauvierre, Chatenay-Malabry (FR); Patrick Couvreur, Villebon-sur-Yvette (FR); Denis Labarre, Villebon-sur-Yvette (FR); Liliane Leclerc, Paris (FR); Michael Marden, Aulinay-sous-Bois (FR)

Correspondence Address: NIXON & VANDERHYE, PC, 901 NORTH GLEBE ROAD, 11TH FLOOR, ARLINGTON, VA, 22203, US

(21) Appl. No.: 10/533,084

(22) PCT Filed: Jun. 10, 2003
(86) PCT No.: PCT/FR03/01435

§ 371 Date: Dec. 5, 2005

(51) Int. Cl. A61K 9/14; A61K 38/42; C07K 14/80

(52) U.S. Cl. 424/489; 530/385; 525/54.1; 977/906

(57) ABSTRACT

The invention relates to novel compounds, characterized in that they comprise a hemoprotein which is associated with a sequenced block copolymer, comprising an hydrophilic segment which is an oligo or polysaccharide linked to at least one hydrophobic segment of formula (1) wherein x represents H or an radical which is alkyl, CN or CONHR, Y represents a COOR′, CONHR″ or C₆H₅ radical, wherein R, R′ and R″ represent, independently from each other, a hydrogen atom, a C₁-C₂₀ linear ou branched alkyl group, a C₁-C₂₀ linear or branched alcoxy group, an amino acid radical, a mono- or poly-hydroxyl acid radical or an aryl ou heteroaryl C₅-C₁₂ radical, and forms associated with a gas. The invention can be used as blood substitutes and depolluting agents.

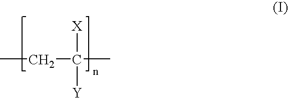

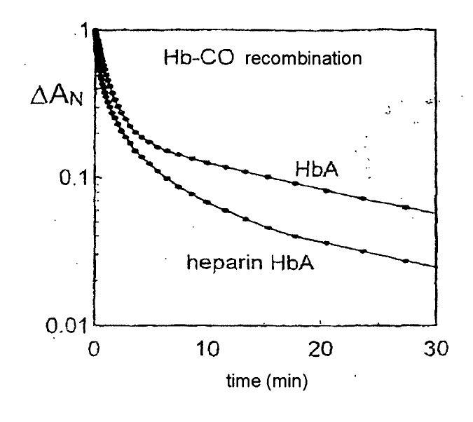

The invention relates to novel compounds based on copolymers with a block structure comprising a hydrophilic segment linked to at least one hydrophobic segment, and to applications thereof in particular for the development of blood substitutes and as depolluting agents.

Many studies have related to the search for products that can be used as blood substitutes to make up for needs associated with emergency situations (natural disasters, road accidents, wars) and with the decrease in blood donors and, in general, in order to avoid possible contamination problems during transfusions.

Among the products currently proposed, mention will be made of perfluorocarbon emulsions and hemoglobin solutions.

Perfluorocarbons are halogenated fatty acids that have the property of increasing oxygen solubility in aqueous medium; hemoglobin solutions consist of polymerized hemoglobin.

However, perfluorocarbons cannot contain satisfactory amounts of oxygen. As regards solutions of normal isolated hemoglobins, that are used in vivo, they result in severe vasoconstriction and undergo irreversible autooxidation. The encapsulation of hemoglobin-based systems has therefore been proposed as a solution to these problems, but it has been found that these capsules are rapidly removed from the blood circulation and that they do not protect the hemoglobin against oxidation.

Now, the inventors have noted that previously developed copolymers, that can be used as active principle vectors, are capable of associating hemoproteins in a general manner, according to amounts of the order of at least 25 mg of hemoglobin per gram of polymer, which gives them great value as oxygen transporters.

The term “hemoprotein” as used in the invention comprises normal hemoproteins, such as cytochromes or myoglobins, and also modified hemoproteins, in particular natural or modified hemoglobins, that are for example bridged, polymerized, mutated or comprise more or less long peptide chains. The invention also extends to hemoprotein analogs in which the iron is substituted with another metal, for example with cobalt, magnesium, copper or zinc.

In addition, advantageously, such substitutes exhibit great stability. A not insignificant amount of the associated hemoprotein molecule in fact remains attached to the copolymer after treatment with surfactants.

The aim of the invention is therefore to provide, as novel products, compounds of said copolymers with hemoproteins.

The invention is also directed toward the applications of these compounds for developing human or animal blood substitutes and their use in particular in various human or veterinary pathological situations, or else as depolluting agents.

The compounds of the invention are characterized in that they comprise a hemoprotein associated with a sequenced block copolymer comprising a hydrophilic segment that is an oligosaccharide or a polysaccharide, linked to at least one hydrophobic segment of formula 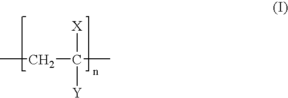
in which:
X represents H or an alkyl, CN or CONHR radical,
Y represents a COOR′, CONHR″ or C₆H₅ radical,
   with R, R′ and R″ representing, independently of one another, a hydrogen atom, a linear or branched C₁ to C₂₀ alkyl group, a linear or branched C_{1 to C}₂₀ alkoxy group, an amino acid radical, a mono- or polyhydroxylated acid radical or a C₅ to C₁₂ aryl or heteroaryl radical, and the forms associated with a gas.

The hemoprotein is natural or modified. It is especially hemoglobin, where appropriate recombinant.

The copolymers are in particular described in application WO 02/39979 published on May 23, 2002, in the name of the CNRS [French National Center for Scientific Research] (inventors, Chauvierre et al.). They are in the form of particles of 1 nm to 1 mm. In these copolymers, said hydrophilic segment is linked, via one of its ends, to a single hydrophobic segment of formula (I), or via each of its two ends, to a hydrophobic segment, the two hydrophobic segments being identical or different.

For biological applications, x preferably represents a CN radical and Y an ester radical. Copolymers that are especially advantageous for the implementation of such applications comprise, as hydrophobic segment, poly(alkyl cyanoacrylate)s. For applications such as depolluting gas, X is advantageously H and Y a phenyl or ester radical.

The hydrophilic segment that is saccharide in nature is a natural or synthetic oligosaccharide or polysaccharide, that may or may not be modified, as defined in application WO 02/39979. It is advantageously dextran, where appropriate sulfated, or heparin.

The copolymers of the invention are in the form of particles of 1 nm to 1 mm. For biological applications, in particular as blood substitutes, the copolymers are in the form of nanoparticles of said compounds.

These nanoparticles can be obtained according to the polymerization technique for assembly by covalent bonding of at least one hydrophobic segment of general formula (I) with a natural or modified oligosaccharide and/or polysaccharide segment, in particular according to the radical polymerization technique described in said application WO 02/39979.

The core of the nanoparticles, consisting of the hydrophobic amorphous polymer, allows the loading of hydrophobic compounds, such as antioxidants, which makes it possible to limit the percentage of formation of methemoglobin.

The structure of the compounds makes it possible to prevent their uptake by the organism's nonspecific immune defense system and, as a result, ensures the prolonged circulation thereof in the bloodstream.

The gas-associated forms of the compounds of the invention are also within the field of the invention. The invention is in particular directed toward associations with oxygen.

The obtaining of the compounds of the invention comprises bringing a colloidal suspension of said nanoparticles into contact with a solution of hemoprotein, for a period of time sufficient to obtain the association of the hemoprotein, advantageously followed by a purification step.

The compounds of the invention do not exhibit any toxicity in humans. It will also be advantageously noted that sizes of the order of a nanometer allow the particles to gain access to the vascular microcirculation. These products are nonimmunogenic, bioerodable and stable.

The invention is therefore directed toward the biological applications of these compounds, most especially as human or animal blood substitutes.

Nanoparticle development technology makes it possible to vary the size of the compounds, but also the composition of the polysaccharides at the surface of the nanoparticles. It is thus possible, from the point of view of a use in transfusion, to choose polysaccharides that have biological properties capable of facilitating or of targeting the supply of oxygen to the tissues concerned. Thus, according to the polysaccharide used, the product will be indicated for treating a hemorrhagic syndrome, an occlusive vascular event, or as an adjuvant to an antitumor therapy, for instance as a radiosensitizing agent. By way of example, vectors coated with heparin have the advantage of associating hemoglobin, while at the same time conserving the anticoagulant properties of heparin. This blood substitute is therefore more particularly suitable for vasoocclusive events.

It will also be noted that the starting materials for developing the substitutes of the invention, and the processes for obtaining them, are relatively inexpensive and that it is possible to produce them in large amounts.

Thus, the invention is of great value in the medical field since the blood substitute market is a worldwide market, there is a continuously increasing demand, and this market is still awaiting a blood substitute that is effective and has no side effects.

The invention is also directed toward the pharmaceutical compositions characterized in that they contain a therapeutically effective amount of at least one compound in the form of nanoparticles as defined above, in combination with a pharmaceutically acceptable vehicle. These compositions will be administered according to dosages that are suitable for the emergency situation and for the pathology to be treated, which will be readily determined by those skilled in the art.

These compositions are provided in the form of injectable solutions. They are more particularly compositions in which the nanoparticles are in a physiological saline.

The invention is also directed toward the use of the compounds as defined above, as agents for depolluting gases, such as carbon monoxide or nitric oxide.

Other characteristics and advantages of the invention will emerge from the following examples, with reference to the single FIGURE that represents the results of flash photolysis.

### EXAMPLE 1

### Nanoparticles Derived from a Copolymer Consisting of Dextran and of Poly(isobutyl cyanoacrylate) (PIBCA)

0.1375 g of dextran having a variable molar mass (15 000 and 71 000 g/mol) are dissolved, in a glass tube 2 cm in diameter, in 8 ml of HNO₃ (0.2 mol/l), with magnetic stirring at 40° C. and with slight bubbling with argon. After 10 minutes, 2 ml of acidic solution of cerium ions (8×10⁻² M of cerium IV ammonium nitrate in HNO₃ at 0.2 mol/l), and then 0.5 ml of isobutyl cyanoacrylate are added. After 10 minutes, the bubbling with argon is stopped and the glass tube is stoppered. After at least 40 minutes, the stirring is stopped and the glass tube is cooled under tap water. The pH is adjusted with NaOH (1 N) so as to directly obtain a value of 7±0.5 after the addition of 1.25 ml of trisodium citrate dihydrate (1.02 M). Finally, the suspension is stored in the cold.

At this stage, a suspension of stable colloidal polymer particles is obtained. The copolymers constituting the particles are purified as follows:

Dialysis bags (Spectra/Por® CE MWCO: 100 000) are regenerated for 30 minutes with osmosed water. The colloidal suspensions, that have been vortexed, are introduced into the regenerated bags.

After two successive dialyses for 1 h 30 min against 5 liters of osmosed water, followed by an overnight dialysis against 5 liters of osmosed water, the purified copolymers, contained in the dialysis bags, are recovered and conserved in the cold (refrigerator).

### EXAMPLE 2

### Nanoparticles Derived from a Copolymer of Heparin and of Poly(isobutyl cyanoacrylate)

The same protocol as that described in example 1 is reproduced, using 0.1375 g of heparin in place of the dextran.

### EXAMPLE 3

### Nanoparticles Derived from a Copolymer of Heparin, of Dextran and of Poly(isobutyl cyanoacrylate)

The same protocol as that described in example 1 is reproduced, using 0.0688 g of heparin and 0.6688 g of dextran in place of the 0.1375 g of dextran.

### EXAMPLE 4

### Nanoparticles Derived from a Copolymer of Dextran Sulfate and of Poly(isobutyl cyanoacrylate)

The same protocol as that described in example 1 is reproduced, using 0.1375 g of dextran sulfate of variable molar mass (10 000 and 40 000 g/mol) in place of the dextran.

### EXAMPLE 5

### Concentration of the Colloidal Suspensions

The colloidal suspensions can optionally be concentrated by ultrafiltration on an Amicon cell equipped with a 300 kD Omega membrane.

### EXAMPLE 6

### Step Consisting in Associating the Hemoglobins with the Various Nanoparticles

The colloidal suspension (1 ml) is brought into contact, overnight, with variable volumes (from 25 to 100 μl) of solution of bridged or normal adult hemoglobin at 100 mg/ml, and equilibrated under 10% carbon monoxide.

The hemoglobin-loaded colloidal suspensions (1 ml) are isolated by filtration on a Sephacryl® S100 column (60 cm long) equilibrated in 100 mM sodium phosphate buffer, pH 7.4. The eluates comprising the nanoparticles are then ultrafiltered on an Amicon cell equipped with a 300 kD Omega membrane and rinsed with 4 ml of solution containing 100 mM sodium phosphate and 150 mM NaCl, pH 7.4. The ultrafiltered nanoparticles are taken up in 1 ml of 100 mM sodium phosphate buffer containing 150 mM NaCl, pH 7.4.

### EXAMPLE 7

### Determination of the Amount of Hemoglobin Associated with the Various Nanoparticles

All the fractions eluted from the S100 gel filtration column that are free of nanoparticles are recovered and mixed, and the total volume is measured. The ultrafiltrates are also recovered and mixed, and the total volume is evaluated. A spectrophotometric assay of the cyanomethemoglobin, read at 540 nm, is then carried out according to Drabkin's method, on all the previously recovered hemoglobin solutions. The amount of hemoglobin associated with the nanoparticles is estimated with respect to a control (solution of hemoglobin of known concentration that has undergone the same analytical treatment).

Table 1 reports the results of the association of hemoglobin with the nanoparticles. The amount of normal human hemoglobin associated with the various nanoparticles is expressed as mg per ml of nanoparticulate suspension.

TABLE 1

| | Amounts of associated |
|---|---|
| | normal human |
| Types of nanoparticles | hemoglobin (mg/ml) |
| Dextran 71 000-PIBCA | 0.84 |
| Dextran 15 000-PIBCA | 1.28 |
| Dextran sulfate 40 000-PIBCA | 1.88 |
| Dextran sulfate 10 000-PIBCA | 1.24 |
| Dextran 71 000 and heparin-PIBCA | 1.07 |
| Heparin-PIBCA | 2.09 |

### EXAMPLE 8

### Determination of the Size of the Various Nanoparticles

A control of the size of the nanoparticles is performed by quasi-elastic light scattering, after synthesis and purification of the latter, and then after binding of the hemoglobins.

The nanoparticle suspensions are diluted in MilliQ® water so that the number of particles per ml is suitable for the measuring device.

The hydrodynamic diameters of the various particles after synthesis, after purification and after association of hemoglobin are given in table 2 below (Hb A: normal human hemoglobin).

TABLE 2

| | Mean hydrodynamic diameters ± standard | | |
|---|---|---|---|
| | deviations over the distribution (nm) | | |
| Types of | After | After | After Hb A |
| nanoparticles | synthesis | purification | association |
| Dextran | 292 ± 71 | 293 ± 47 | 305 ± 86 |
| 71 000-PIBCA | | | |
| Dextran | 197 ± 46 | 202 ± 42 | 197 ± 50 |
| 15 000-PIBCA | | | |
| Dextran sulfate | 267 ± 40 | 274 ± 64 | 244 ± 41 |
| 40 000-PIBCA | | | |
| Dextran sulfate | 185 ± 45 | 192 ± 47 | 170 ± 40 |
| 10 000-PIBCA | | | |
| Heparin-PIBCA | 103 ± 34 | 110 ± 42 | 104 ± 36 |

### EXAMPLE 9

### Functional Studies of the Hemoglobins Associated with the Nanoparticles

The dynamic properties of a functional hemoglobin are controlled in the hemoglobin CO form (after reduction with dithionite and association of carbon monoxide at 10%) by flash photolysis and by means of the static spectral properties between 710 nm and 380 nm.

The single FIGURE reports the differences in absorbance ΔA_{N} as a function of time. The hemoglobin CO associated with the various types of nanoparticles studied conserves a normal spectrum with its characteristic absorbance peaks at 420, 540 and 576 nm. From a functional point of view, the hemoglobin associated with the nanoparticles shows a reversible ligand-binding capacity, which property is essential for its oxygen transporter role.

### EXAMPLE 10

### Determination of the Surface Charges of the Hemoglobin-Loaded Nanoparticles

The suspensions of hemoglobin-loaded nanoparticles are diluted to 1/200th in a 1 mM NaCl solution, and are then analyzed using a zeta-meter.

The zeta potentials of the various particles before and after association of the hemoglobin are given in table 3 below (Hb A: normal human hemoglobin).

TABLE 3

Zeta potentials ±
standard deviation (mV)

| Types of | Before Hb A | After Hb A |
|---|---|---|
| nanoparticles | association | association |
| Dextran | −11 ± 2 | −6 ± 2 |
| 71 000-PIBCA | | |
| Dextran | −19 ± 2 | −17 ± 2 |
| 15 000-PIBCA | | |
| Dextran sulfate | −42 ± 2 | −45 ± 2 |
| 40 000-PIBCA | | |
| Dextran sulfate | −43 ± 2 | −44 ± 2 |
| 10 000-PIBCA | | |
| Heparin-PIBCA | −48 ± 2 | −44 ± 2 |

### EXAMPLE 11

### Studies of the Function of the Polysaccharides on the Surface of the Nanopaticles After the Hemoglobin-Loading Thereof

The hemoglobin-loaded nanoparticulate suspensions exhibiting heparin at their surface are subjected to the von Willebrand factor-binding test.

The properties of recognition of the heparin by the von Willebrand factor are not impaired.

1. A novel compound, characterized in that it comprises a hemoprotein associated with a sequenced block copolymer comprising a hydrophilic segment, that is an oligosaccharide or a polysaccharide, linked to at least one hydrophobic segment of formula 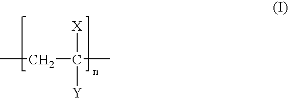
in which:
X represents H or an alkyl, CN or CONHR radical,
Y represents a COOR′, CONHR″ or C₆H₅ radical,
with R, R′ and R″ representing, independently of one another, a hydrogen atom, a linear or branched C₁ to C₂₀ alkyl group, a linear or branched C₁ to C₂₀ alkoxy group, an amino acid radical, a mono- or polyhydroxylated acid radical or a C₅ to C₁₂ aryl or heteroaryl radical, and the forms associated with a gas.

2. The novel compound as claimed in claim 1, characterized in that the hemoprotein is a normal hemoprotein, such as cytochromes or myoglobins, or a modified hemoprotein, in particular a natural or modified hemoglobin, that is for example bridged, polymerized, mutated or comprises more or less long peptide chains, or else a hemoprotein analogue in which the iron is substituted with another metal, for example with cobalt, magnesium, copper or zinc.

3. The compound as claimed in claim 1, characterized in that the hemoprotein is a normal or modified hemoglobin.

4. The compound as claimed in claim 1, characterized in that, in formula (I), X represents a CN radical.

5. The compound as claimed in claim 4, in which the hydrophobic segment is a poly(alkyl cyanoacrylate).

6. The compound as claimed in claim 1, characterized in that the hydrophilic segment that is saccharide in nature is a natural or synthetic oligosaccharide or polysaccharide, that may or may not be modified, in particular dextran, where appropriate sulfated, or heparin.

7. The compound as claimed in claim 1, characterized in that X represents H and Y a phenyl or ester radical.

8. The compound as claimed in claim 1, characterized in that it is provided in the form of particles of 1 nm to 1 mm.

9. The compound as claimed in claim 8, characterized in that it is provided in the form of nanoparticles.

10. The use of the compound as claimed in claim 9, as a human or animal blood substitute.

11. The use of the compound as claimed in claim 10, as an adjuvant for antitumor compositions or other antitumor means, for example as a radiosensitizing agent.

12. The use of the compound as claimed in claim 1, as agent for depolluting gases, such as carbon monoxide or nitric oxide.

13. A pharmaceutical composition, characterized in that it contains a therapeutically effective amount of at least one compound as claimed in claim 1, in the form of nanoparticles in combination with a pharmaceutically acceptable vehicle.
